(19) United States
(12) Patent Application Publication
(10) Pub. No.: US 2021/0236443 A1
(43) Pub. Date: Aug. 5, 2021

(54) METHOD OF TREATMENT OF NON-ALCOHOLIC STEATOHEPATITIS, NASH

(71) Applicant: KRISH BIOTECH RESEARCH PRIVATE LIMITED, Kolkata (IN)

(72) Inventors: Raghuram ANUPINDI, Maharashtra (IN); Raghib HUSAIN, Maharashtra (IN); Atul CHURIWAL, Kolkata (IN)

(73) Assignee: KRISH BIOTECH RESEARCH PRIVATE LIMITED, Kolkata (IN)

(21) Appl. No.: 17/049,293

(22) PCT Filed: Apr. 18, 2019
(86) PCT No.: PCT/IN2019/050317

§ 371 Date: Oct. 20, 2020

Related U.S. Application Data
(60) Provisional application No. 62/660,601, filed on Apr. 20, 2018.

(51) Int. Cl. A61K 31/192; A61K 31/381; A61K 31/426; A61K 31/428; A61K 31/155; C07C 59/72; C07D 333/16; C07D 277/24; C07D 277/60; A61P 1/16; A61K 45/06; A61K 9/00

(52) U.S. Cl. CPC A61K 31/192; A61K 31/381; A61K 31/426; A61K 31/428; A61K 31/155; A61K 9/0053; C07D 333/16; C07D 277/24; C07D 277/60; A61P 1/16; A61K 45/06; C07C 59/72

(57) ABSTRACT

There is disclosed a method of treating a subject suffering from non-alcoholic steatohepatitis (NASH), non-alcoholic fatty liver disease (NAFLD), steatosis, lobular inflammation, or liver fibrosis, comprising administering to said subject a therapeutically effective amount of at least one of the compounds of General Formula (I) or a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere thereof;

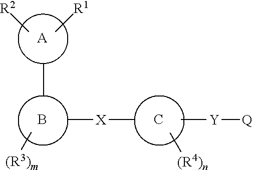

wherein A, B, C, R¹, R², R³, R⁴, X, Y, Q, m, n are as defined herein.

### RELATED APPLICATION

This application claims priority from U.S. provisional application No. 62/660,601, the content of which is referred herein in its entirety.

### FIELD OF INVENTION

The invention relates in general to methods of treating non-alcoholic steatohepatitis (NASH), symptoms and manifestations thereof, and other related liver disorders.

### BACKGROUND OF THE INVENTION

NAFLD and NASH are increasingly common forms of chronic liver disease, e.g., with hepatic steatosis as a common pathological feature. In NASH, hepatic steatosis is associated with hepatic inflammation. Other terms that have been used to describe NASH include pseudoalcoholic hepatitis, alcohol-like hepatitis, fatty liver hepatitis, steatonecrosis, and diabetic hepatitis.

NAFLD is a chronic liver disease, affecting 2.8-24% of the general population all over the world. It is increasingly diagnosed worldwide and considered to be the commonest liver disorder in Western countries, affecting approximately 15-30% of the general population, and its prevalence increases steadily to 70-90% in people with obesity or type 2 diabetes. It is also currently a common cause of abnormal liver function and is recognized as a major cause of cryptogenic cirrhosis of liver.

NASH can be defined as a chronic liver disease with inflammation caused by fat accumulation in the absence of alcohol consumption (<20 gr/day). NASH may progress to cirrhosis and hepatocellular cancer in about 5% of patients (Curr Opin Gastroenterol. vol 33, 2017 pp. 134-141). Currently, there are no drugs that are approved by FDA for treating NASH. In the absence of proper treatments, the following approaches are being followed:
1. Life style modifications, such as diet and exercise;
2. Experimental therapies, such as use of lipid lowering and anti-obesity agent, metformin, thiazolidinediones, angiotensin receptor blockers and n-3 PUFAs, anti-oxidant (Vitamin E), pentoxifylline, etc; and
3. Bariatric surgery is considered in patients when the above approaches fail and must be performed before the patient develops cirrhosis. Liver transplantation is successful in liver failure patients, but NASH may recur after transplantation and is likely to be denied to patients with morbid obesity.

However, each of these approaches have drawbacks and none are able to effectively address the causes of NASH.

### SUMMARY OF THE INVENTION

Provided herein is a method of treating a subject suffering from non-alcoholic steatohepatitis (NASH), comprising administering a therapeutically effective amount of a compound of General Formula (I), which is shown herein below, or pharmaceutically acceptable salt thereof, to the subject. Also provided herein is a method of treating a subject suffering from non-alcoholic fatty liver disease (NAFLD), comprising administering a therapeutically effective amount of a compound of General Formula (I) or a pharmaceutically acceptable salt thereof to the subject. The disclosure also provides a method of slowing the progression of NAFLD to NASH in a subject in need thereof, comprising administering a therapeutically effective amount of a compound of General Formula (I) or a pharmaceutically acceptable salt thereof to the subject. The disclosure also provides a method of reducing liver inflammation in a subject suffering from non-alcoholic steatohepatitis (NASH), comprising administering a therapeutically effective amount of a compound of General Formula (I) or a pharmaceutically acceptable salt thereof to the subject. The disclosure also provides a method of treating steatosis in a subject suffering therefrom comprising administering to the subject a therapeutically effective amount of a compound of General Formula (I) or a pharmaceutically acceptable salt thereof. The disclosure also provides a method of treating lobular inflammation in a subject suffering therefrom comprising administering to the subject a therapeutically effective amount of a compound of General Formula (I) or a pharmaceutically acceptable salt thereof. The disclosure also provides a method of treating liver fibrosis in a subject suffering therefrom comprising administering to the subject a therapeutically effective amount of a compound of General Formula (I) or a pharmaceutically acceptable salt thereof.

In some embodiments, compounds utilized herein include a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere of a General Formula (I).

In some embodiments, the compound administered is administered as a pharmaceutical composition. The pharmaceutical composition comprises at least one pharmaceutically acceptable excipient.

In some embodiments, the composition further comprises at least one known compound such as metformin, a thiazolidinedione, PPAR alpha/delta agonists, FXR agonists, and PPAR alpha/gamma agonists.

In some embodiments, the composition is administered orally. In some embodiments, the composition is administered parenterally.

Also provided herein is a method of diagnosing and treating non-alcoholic steatohepatitis (NASH) in a patient, said method comprising:

a. obtaining a liver tissue sample from a human patient;

b. detecting concurrent necroinflammatory reactions of the liver and hepatocellular ballooning with or without fibrosis and/or cirrhosis by way of biopsy;

c. diagnosing the patient suffering from NASH when concurrent necroinflammatory reactions of the liver and hepatocellular ballooning with or without fibrosis and/or cirrhosis are detected; and

d. administering an effective amount of compound of General Formula (I) or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof to the diagnosed patient.

Also provided herein is a compound of General Formula (I) or a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere thereof for use in the treatment of a patient suffering from non-alcoholic steatohepatitis (NASH), or another disorder disclosed herein.

Also provided herein is a use of a compound of General Formula (I) or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof for preparation of a medicament for treatment of non-alcoholic steatohepatitis (NASH), or another disorder disclosed herein.

The disclosure also provides a use of a compound of General Formula (I), in preparation of a medicament for treatment of NASH and other disorders as disclosed herein. The disclosure also provides a use of a compound of General Formula (I) in preparation of a medicament for administration in combination with a second agent for treating NASH and other disorders as disclosed herein. Also provided is use of a second agent for treating NASH, and other disorders as disclosed herein, in preparation of a medicament for administration in combination with a compound of General Formula (I). Further provided are kits comprising a compound or composition of one or more compound of General Formula (I) for treatment of NASH.

### BRIEF DESCRIPTION OF THE ACCOMPANYING DRAWINGS

FIG. 1 shows a Histology Activity Index (HAI-Knodell score) of compounds SI. No. 8: (4-(4-((2-(5-Cyclopropylthiophen-2-yl)-S-fluorobenzyl)oxy)phenyl)butanoic acid; and SI. No. 25: (4-(4-((5-Fluoro-2-(4,5,6,7-tetrahydrobenzo[d]thiazol-2-yl)benzyl)oxy)phenyl)butanoic acid); as identified hereinafter.

FIG. 2 shows the Liver histology of animals treated with compounds SI. No. 8 and 25 as identified hereinafter.

### DETAILED DESCRIPTION OF THE INVENTION

As disclosed herein, the present invention utilizes a compound of General Formula (I),

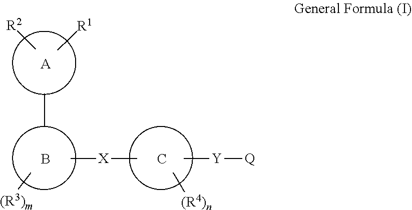

or a tautomer, a stereoisomer or a geometrical isomer thereof; or pharmaceutically acceptable salt, a pharmaceutically acceptable solvate, a prodrug, a polymorph, an N-oxide, a S-oxide or a carboxylic acid isostere thereof;

wherein:

Ring A is a saturated or unsaturated 4- to 10-membered carbocycle; a 5- to 10-membered heteroaryl; or a saturated or partly saturated or unsaturated 5- to 10-membered heterocycle; wherein said heteroaryl or heterocycle contain 1, 2, 3 or 4 heteroatoms independently selected from N, O and S;

Ring B and Ring C are independently selected from the group consisting of (C₆-C₁₀) aryl and 6- to 10-membered heteroaryl which contains 1, 2 or 3 heteroatoms independently selected from the group consisting of N, O and S;

X is —(CR⁸R⁹)ₚ—O—, —(CR⁸R⁹)ₚ—S—, —(CR⁸R⁹)ₚ—N(R¹⁰)—, —O—(CR⁸R⁹)ₚ—, —S—(CR⁸R⁹)ₚ— or —N(R¹⁰)—(CR⁸R⁹)ₚ;

Y is —(CR¹⁴R¹⁵)_{g}—;

Q is —CO₂M, —CONH₂, —CONH[(C₁-C₆)alkyl], —CON[(C₁-C₆)alkyl]₂ or —CONHSO₂(C₁-C₆)alkyl; M is hydrogen, deuterium or (C₁-C₆)alkyl;

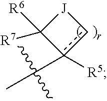

R¹ is

wherein

[symbol] is point of attachment to ring A;

J is —CH₂—, —CHF—, —CF₂—, —CH[(C₁-C₆)alkyl]-, —C[(C₁-C₆)alkyl]₂-, —O—, —NR^{a}— or —S—;

“ [symbol] ” represents an optional bond;

R^{a} is hydrogen, (C₁-C₆)alkyl or halo(C₁-C₆)alkyl;

R² is selected from the group consisting of hydrogen, halogen, (C₁-C₆)alkyl, halo(C₁-C₆)alkyl, (C₁-C₆)alkoxy, hydroxy, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, (C₆-C₁₀)aryloxy, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³;

or

R¹ and R² are combined together with one or two atoms of Ring A to form:
i) a 3- to 8-membered partly unsaturated or saturated carbocycle; or
ii) a 4- to 8-membered saturated heterocycle which contains 1, 2 or 3 heteroatoms independently selected from the group consisting of N, O and S;

wherein the said carbocycle or heterocycle can be unsubstituted or substituted with one or more groups independently selected from the group consisting of (C₁-C₆)alkenyl, (C₁-C₆)alkyl, halo(C₁-C₆)alkyl, (C₁-C₆)alkoxy, hydroxy, halogen, cyano, oxo, (C₃-C₁₀)cycloalkyl, (C₆-C₁₀)aryl, heteroaryl and heterocyclyl;

R³ at each occurrence, is independently selected from the group consisting of hydrogen, halogen, (C₁-C₆)alkyl, halo(C₁-C₆)alkyl, (C₁-C₆)alkoxy, hydroxy, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, (C₆-C₁₀)aryloxy, heterocyclyl-(C₁-C₆)alkyl, (C₁-C₆)alkyl-heterocyclyl-, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³;

R⁴, at each occurrence, is independently selected from the group consisting of hydrogen, (C₁-C₆)alkyl, halo(C₁-C₆)alkyl, (C₁-C₆)alkoxy, hydroxy, halogen, cyano, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, (C₆-C₁₀)aryloxy, heterocyclyl-(C₁-C₆)alkyl, (C₁-C₆)alkyl-heterocyclyl, heteroaryl-(C₁-C₆)alkyl, (C₁-C₆)alkyl-heteroaryl, —NR¹⁰R¹¹, —S(O)ₜR¹² and —C(O)R¹³;

R⁵ is selected from the group consisting of hydrogen, (C₁-C₆)alkyl, hydroxy, cyano, —COR¹⁰, —NR¹⁰R¹¹, —CONR¹⁰R¹¹, (C₁-C₆)alkylamino, di(C₁-C₆)alkylamino, (C₁-C₆)alkoxy, halo(C₁-C₆)alkyl, —S(O)ₜR¹² and —C(O)R¹³;

R⁶ and R⁷ are independently selected from the group consisting of hydrogen, (C₁-C₆)alkyl, halo(C₁-C₆)alkyl and halogen;

R⁸ and R⁹ are independently selected from the group consisting of hydrogen, deuterium, (C₁-C₆)alkyl, halo(C₁-C₆)alkyl and halogen; or

R⁸ and R⁹ can combine together to form
i) a 3- to 5-membered saturated carbocycle selected from the group consisting of cyclopropane, cyclobutane, cyclopentane and cyclohexane; or
ii) a 4- to 6-membered saturated heterocycle selected from the group consisting of oxetane, thietane, azetidine, tetrahydrofuran, tetrahydrothiophene, pyrrolidine and piperidine;

R¹⁰ is hydrogen, hydroxy, (C₁-C₆)alkyl, halo(C₁-C₆)alkyl, (C₁-C₆)alkoxy, (C₃-C₁₀)cycloalkyl, (C₆-C₁₀)aryl, heterocyclyl, heteroaryl, (C₆-C₁₀)aryloxy, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl or (C₁-C₆)alkyl-heteroaryl or —S(O)ₜR¹²;

R¹¹ is hydrogen, hydroxy, (C₁-C₆)alkyl, halo(C₁-C₆)alkyl, (C₃-C₁₀)cycloalkyl, (C₁-C₆)alkoxy, (C₃-C₁₀)cycloalkyl, (C₆-C₁₀)aryl, heterocyclyl, heteroaryl, (C₆-C₁₀)aryloxy, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl or —S(O)ₜR¹²; or

R¹⁰ and R¹¹ are combined together to form a 3- to 8-membered saturated or unsaturated ring which contains 1, 2 or 3 heteroatoms independently selected from the group consisting of N, O and S;

R¹² and R¹³ are independently selected from the group consisting of hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl and (C₁-C₆)alkyl-heteroaryl;

R¹⁴ and R¹⁵ are independently selected from the group consisting of hydrogen, (C₁-C₆)alkyl, halo(C₁-C₆)alkyl and halogen;

or

R¹⁴ and R¹⁵ are combined together to form a 3- to 5-membered saturated carbocycle or 4- to 6-membered saturated heterocycle which optionally contains 1 or 2 heteroatoms independently selected from the group consisting of N, O and S; wherein the said carbocycle or heterocycle can be substituted or unsubstituted.

g is 2, 3, 4, 5 or 6;

m is 0, 1 or 2;

n is 0, 1 or 2;

p is 1, 2 or 3;

r is 0, 1, 2, 3 or 4;

t is 0, 1 or 2;

wherein

(C₁-C₆)alkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, (C₆-C₁₀)aryloxy, heterocyclyl, heteroaryl, amino, cyano, nitro, —NH(C₁-C₆)alkyl, —N[(C₁-C₆)alkyl]₂, —C(O)(C₁-C₆)alkyl, —C(O)O(C₁-C₆)alkyl, —C(O)NH₂, —C(O)NH(C₁-C₆)alkyl, —C(O)N[(C₁-C₆)alkyl]₂ and —C(O)NHSO₂(C₁-C₆)alkyl;

(C₃-C₁₀)cycloalkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, amino, cyano and nitro;

carbocycle is unsubstituted or substituted with one or more groups independently selected from the group consisting of (C₁-C₆)alkyl, halo(C₁-C₆)alkyl, hydroxy, halogen, (C₁-C₆)alkoxy, halo(C₁-C₆)alkoxy, (C₆-C₁₀)aryl, (C₃-C₁₀)cycloalkyl, heteroaryl, heterocyclyl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;

(C₆-C₁₀)aryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;

heterocyclyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)NR¹⁰R¹¹ and —S(O)ₜR¹²; wherein R¹⁰, R¹¹, R¹² and t are as defined above;

heteroaryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, heterocyclyl, heteroaryl, amino, cyano, nitro, —C(O)NR¹⁰R¹¹ and —S(O)ₜR¹²; wherein R¹⁰, R¹¹, R¹² and t are as defined above.

In some embodiments, compounds utilized herein include a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere of a General Formula (I).

Before disclosing the invention in further details, the following definitions are provided.

### Definitions

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

The singular forms “a”, “an” and “the” include plural aspects unless the context clearly dictates otherwise. For instance, the terms “a”, “an” and “the” refers to “one or more” when used in the subject specification, including the claims. Thus, for example, reference to “a compound” may include a plurality of such compounds, or reference to “a disease” or “a disorder” includes a plurality of diseases or disorders.

It should also be noted that the term “or” is generally employed in its sense including “and/or” unless the content clearly dictates otherwise.

The term “independently” when used in the context of selection of substituents for a variable, it means that where more than one substituent is selected from a number of possible substituents, those substituents may be the same or different.

As used herein, the term “(C₁-C₆)alkyl” or “alkyl” as used herein; alone or as part of a substituent group, refers to the radical of saturated aliphatic groups, including straight or branched-chain alkyl groups. A straight-chain or branched chain alkyl has six or fewer carbon atoms in its backbone, for instance, C₁-C₆ for straight chain and C₃-C₆ for branched chain. As used herein, (C₁-C₆)-alkyl refers to an alkyl group having 1 to 6 (both inclusive) carbon atoms; preferably refers to an alkyl group having 1 to 4 (both inclusive) carbon atoms i.e. (C₁-C₄)-alkyl. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl. In the “(C₁-C₆)alkyl” group, one or more carbon atoms can be optionally replaced with one or more heteroatoms independently selected from N, O and S.

Furthermore, unless stated otherwise, the alkyl group can be unsubstituted or substituted with one or more groups; preferably with 1-4 groups, independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, (C₆-C₁₀)aryloxy, heterocyclyl, heteroaryl, amino, cyano, nitro, —NH(C₁-C₆)alkyl, —N [(C₁-C₆)alkyl]₂, —C(O)(C₁-C₆)alkyl, —C(O)O(C₁-C₆)alkyl, —C(O)NH₂, —C(O)NH(C₁-C₆)alkyl, —C(O)N[(C₁-C₆)alkyl]₂ and —C(O)NHSO₂(C₁-C₆)alkyl.

As used herein, the term “halo(C₁-C₆)alkyl” or “haloalkyl” refers to the alkyl group which is substituted with one or more halogens. A monohalo(C₁-C₆)alkyl radical, for example, can have a chlorine, bromine, iodine or fluorine atom. Dihalo and polyhalo(C₁-C₆)alkyl radicals can have two or more of the same or different halogen atoms. Representative examples of halo(C₁-C₆)alkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like groups.

As used herein, the term “(C₁-C₆)-alkoxy” or “alkoxy” refers to a (C₁-C₆)-alkyl having an oxygen radical attached thereto. The term “(C₁-C₆)-alkoxy” or “O—(C₁-C₆)-alkyl” or alkoxy wherever used in this specification have the same meaning. Representative examples of alkoxy groups include, but are not limited to, methoxy, ethoxy, propoxy, isopropoxy, n-butoxy, isobutoxy and t-butoxy. Furthermore, unless stated otherwise, the alkoxy groups can be unsubstituted or substituted with one or more groups. A substituted alkoxy refers to a (C₁-C₆)-alkoxy substituted with 1-5 groups, preferably with 1-3 groups selected from the groups indicated above as the substituents for the alkyl group.

As used herein, the term “halogen” refers to chlorine, fluorine, bromine or iodine and is preferably, chlorine, bromine or fluorine.

The term “carbocycle” or “carbocyclic ring” refers to a saturated, partially unsaturated, unsaturated or aromatic 3 to 12 membered monocyclic or bicyclic ring systems whose ring atoms are all carbon, and that the said carbocycle has a single point of attachment to the rest of the molecule. If the carbocycle is a bicyclic ring system, then any one ring in the said bicyclic ring system is a 3-7 membered ring. Representative examples of carbocycle include, but are not limited to, cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. “Aromatic carbocycle” or “aromatic carbocyclic ring” refers to 3 to 12 membered monocyclic or bicyclic aromatic ring systems whose all ring atoms are carbon.

The term “(C₆-C₁₄)-aryl” or “aryl” as used herein refers to monocyclic or bicyclic hydrocarbon groups having 6 to 14 ring carbon atoms, preferably 6 to 10 carbon atoms i.e. “(C₆-C₁₀)-aryl” in which the carbocyclic ring(s) present have a conjugated pi electron system, which may be optionally substituted by one or more groups. Representative examples of (C₆-C₁₄)-aryl include, but are not limited to, phenyl, naphthyl, fluorenyl and anthracenyl.

Furthermore, unless stated otherwise, the aryl group can be unsubstituted or substituted with one or more groups. A substituted aryl refers to a (C₆-C₁₄)-aryl substituted with one or more groups, preferably 1 to 7 groups and more preferably 1 to 3 groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above.

As used herein, the terms “heterocycle”, “heterocyclyl” or “heterocyclic” whether used alone or as part of a substituent group, refers to a 3- to 12-membered, preferably 5- to 10-membered saturated or partially unsaturated monocyclic, bicyclic or tricyclic ring system containing 1 to 4 heteroatoms independently selected from oxygen, nitrogen and sulfur. Saturated heterocyclic ring systems do not contain any double bond, whereas partially unsaturated heterocyclic ring systems, can contain at least one double bond, but do not form an aromatic system containing a heteroatom. The oxidized form of the ring nitrogen and sulfur atom contained in the heterocyclyl to provide the corresponding N-oxide, S-oxide or 5,5-dioxide is also encompassed in the scope of the present invention. Representative examples of heterocyclyls include, but are not limited to, azetidinyl, acridinyl, benzodioxolyl, benzodioxanyl, dioxolanyl, indolizinyl, perhydroazepinyl, phenothiazinyl, phenoxazinyl, tetrahydroisoquinolinyl, piperidinyl, piperazinyl, homopiperazinyl, 2-oxoazepinyl, azepinyl, 4-piperidonyl, pyrrolidinyl, oxazolinyl, oxazolidinyl, isoxazolidinyl, thiazolinyl, thiazolidinyl, isothiazolyl, quinuclidinyl, isothiazolidinyl, indolinyl, isoindolinyl, octahydroindolyl, octahydroisoindolyl, decahydroisoquinolyl, benzopyranyl, morpholinyl, thiomorpholinyl, thiomorpholinyl sulfoxide, tetrahydrofuryl, tetrahydropyranyl, chromanyl and isochromanyl. When the heterocyclyl group represents “5- to 10-membered heterocyclyl”, the representative examples include, but are not limited to, benzodioxolyl, benzo[d][1,3]dioxolyl, benzodioxanyl, tetrahydroisoquinolinyl, piperidinyl, piperazinyl, homopiperazinyl, 2-oxoazepinyl, 4-piperidonyl, pyrrolidinyl, oxazolinyl, oxazolidinyl, and morpholinyl.

Furthermore, unless stated otherwise, the heterocyclyl groups can be unsubstituted or substituted with one or more groups, preferably with 1-7 groups, more preferably with 1-3 groups independently selected from the group consisting of (C₁-C₆)alkyl, halogen, halo(C₁-C₆)alkyl, hydroxy, (C₁-C₆)alkoxy, halo(C₁-C₆)alkoxy, amino, cyano, nitro, —C(O)NR¹⁰R¹¹ and —S(O)ₜR¹²; wherein R¹⁰, R¹¹, R¹² and t are as defined above.

As used herein, the term “heteroaryl” whether used alone or as part of a substituent group, refers to 5- to 10-membered heterocyclyl having an aromatic ring containing one to four identical or different heteroatoms independently selected from oxygen, nitrogen and sulfur atom. Representative examples of heteroaryls include, but are not limited to, pyrrole, pyrazole, imidazole, tetrazole, pyrazine, furan, thiophene, oxazole, oxadiazole, thiazole, benzimidazole, benzoxazole, triazole, benzothiazole, benzofuran, indole, isoindole, cinnoline, indazole, isoindole, thiadiazole, isoquinoline, benzoxazole, thiophene, benzothiazole, isoxazole, triazine, purine, pyridine, quinoline, isoquinoline, phenazine, oxadiazole, pteridine, carbazole, pyridazine, quinazolinyl, pyrimidine, isothiazole, quinoxaline (benzopyrazine), tetrazole, pyrido[2,3-b]pyrazine. The oxidized form of the ring nitrogen and sulfur atom contained in the heteroaryl to provide the corresponding N-oxide, S-oxide or S,S-dioxide is also encompassed in the scope of the present invention.

Furthermore, unless stated otherwise, the heteroaryl groups can be unsubstituted or substituted with one or more groups; preferably with 1-7 groups, more preferably with 1-3 groups independently selected from the group consisting of (C₁-C₆)alkyl, halogen, halo(C₁-C₆)alkyl, hydroxy, (C₁-C₆)alkoxy, halo(C₁-C₆)alkoxy, heterocyclyl, heteroaryl, amino, cyano, nitro, —C(O)NR¹⁰R¹¹ and —S(O)ₜR¹²; wherein R¹⁰, R¹¹, R¹² and t are as defined above. The representative examples of heteroaryl include, but are not limited to, pyrrole, pyrazole, imidazole, pyrazine, furan, thiophene, triazole, benzothiazole, benzofuran, indole, purine, pyridine, quinoline, isoquinoline, pyridazine, quinazolinyl, pyrimidine and isothiazole.

As used herein, the term “(C₃-C₁₂)-cycloalkyl” or “cycloalkyl”, whether used alone or as part of a substituent group, refers to a saturated cyclic hydrocarbon radical including 1, 2 or 3 rings and including a total of 3 to 12 carbon atoms forming the rings, which may be optionally substituted by one or more substituents. The term cycloalkyl includes bridged, fused and spiro ring systems. As used herein, (C₃-C₁₂)-cycloalkyl refers to a cycloalkyl group having 3 to 12 (both inclusive) carbon atoms; preferably, refers to cycloalkyl group having 3 to 10 (both inclusive) carbon atoms i.e. (C₃-C₁₀)-cycloalkyl; and more preferably, refers to cycloalkyl group having 3 to 7 (both inclusive) carbon atoms i.e. (C₃-C₇)-cycloalkyl. Representative examples of cycloalkyl include, but are not limited to, cyclopropyl, cyclobutyl, cyclopentyl, cyclohexyl, perhydronaphthyl, adamantyl, homoadamantyl, noradamantyl, norbornyl, bicyclo[2.1.0]pentanyl, bicyclo[2.2.1]heptyl, spiro[3.3]heptanyl and spiro [4.4]non-2-yl.

The term “tautomer” refers to the coexistence of two or more compounds that differ from each other only in the position of one (or more) mobile atoms and in electron distribution. In fact, tautomers are structural isomers of different energies which are interconvertible via a low energy barrier. For example, proton tautomers (also known as prototropic tautomers) include interconversions via migration of a proton, such as keto-enol and imine-enamine isomerizations.

The term “prodrugs” as used herein refers to any pharmacologically inactive or less active compound which, when metabolized or chemically transformed in vivo by a chemical or physiological process, e.g., a prodrug on being brought to the physiological pH or through enzyme action is converted to the parent compound e.g. the compound of formula (I) of the present invention. For example, in the context of the present invention prodrugs can be esters of the compound of formula (I) which on metabolism the ester group is cleaved to form the active compound of formula (I). Examples of esters include lower alkyl esters, such as the methyl or ethyl ester; carboxy-lower alkyl esters, such as the carboxymethyl ester; nitrooxy- or nitrosooxy-lower alkyl esters, such as the 4-nitrooxybutyl or 4-nitrosooxybutyl ester; and the like.

The phrase, “carboxylic acid isostere” refers to a functional group or a moiety that elicits similar physical, biological and/or chemical properties as a carboxylic acid moiety.

As used herein, the term “pharmaceutically acceptable” means that the carrier, diluent, excipient, and/or salt must be compatible with the other ingredients of the formulation, and not deleterious to the recipient thereof.

The term “pharmaceutically acceptable salt(s)” as used herein includes a salt or salts of the active compound i.e. the compound of formula (I), which retain the desired biological activity of the subject compound and exhibit minimal undesired toxicological effects; and are prepared with suitable acids or bases, depending on the particular substituents found on the compounds described herein.

Within the context of the present invention and as used herein “N-oxide” refers to the oxide of the nitrogen atom of a nitrogen-containing heteroaryl or heterocycle. N-oxide can be formed in the presence of an oxidizing agent for example peroxide such as m-chloro-perbenzoic acid or hydrogen peroxide. N-oxide refers to an amine oxide, also known as amine-N-oxide, and is a chemical compound that contains N→O bond.

Within the context of the present invention and as used herein “S-oxide” refers to the oxide of the sulfur atom (S-oxide) or dioxide of the sulfur atom (S,S-dioxide) of a sulfur-containing heteroaryl or heterocycle. S-oxide and S,S-dioxides can be formed in the presence of an oxidizing agent for example peroxide such as m-chloro-perbenzoic acid or oxone.

Within the context of the present invention and as used herein, the term “solvate” or “solvates” describe a complex wherein the compound of Formula (I) of the present invention, is coordinated with a proportional amount of a solvent molecule. Specific solvates, wherein the solvent is water, are referred to as hydrates.

Within the context of the present invention and as used herein the term “polymorph” or “polymorphic form” or “polymorphs” refer to crystals of the same compound that differs only in the arrangement and/or conformation of the molecule in the crystal lattice.

The term “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young. The term mammal includes animals such as cat, dog, rabbit, bear, fox, wolf, monkey, deer, mouse, pig and human.

As used herein, the terms “treatment” “treat” and “therapy” and the like refer to alleviate, slow the progression, attenuation, or as such treat the existing diseases or condition (e.g. NASH). Treatment also includes treating, or alleviating to some extent, one or more of the symptoms of the diseases or condition.

The term “prophylaxis”, “prevention” or “preventing” can be used interchangeably and mean preventing the disease or disorder by causing the clinical symptoms of the conditions, diseases, disorders or syndromes to not develop or decreasing the development of the disease or disorder or preventing the further development of the disease or disorder in the subjects (the patients).

The term “compound(s) for use” as used herein embrace any one or more of the following: (1) use of compound(s), (2) method of use of compound(s), (3) use in the treatment of, (4) the use for the manufacture of pharmaceutical composition/medicament for treatment/treating or (5) method of treatment/treating/preventing/reducing/inhibiting comprising administering an effective amount of the active compound to a subject in need thereof.

The term, “therapeutically effective amount” as used herein means an amount of a compound of formula (I) or a pharmaceutically acceptable salt thereof or a composition comprising a compound of formula (I) or a salt thereof, effective in producing the desired therapeutic response in a particular patient (subject) suffering from a liver disorder such as NAFLD or NASH. The term “therapeutically effective amount” includes the amount of a compound (in the context of the present invention, the compound of formula (I) or a pharmaceutically acceptable salt thereof), when administered that induces a positive modification in the disease or disorder to be treated or is sufficient to prevent development of, or alleviate to some extent one or more of the symptoms of the disease or disorder being treated in a subject. In respect of the therapeutic amount of the compound, consideration is also given that the amount of the compound used for the treatment of a subject is low enough to avoid undue or severe side effects, within the scope of sound medical judgment. The therapeutically effective amount of the compound or composition will vary with the particular condition being treated, the age and physical condition of the subject, the severity of the condition being treated or prevented, the duration of the treatment, the nature of concurrent therapy, the specific compound or composition employed, the particular pharmaceutically acceptable carrier utilized and other related factors.

Within the context of the present invention and as used herein interchangeably throughout this application, the terms “compounds of General Formula (I)”, and “compounds of the present invention” include all the stereoisomeric and tautomeric forms and mixtures thereof in all ratios, and their pharmaceutically acceptable salts, solvates, polymorphs, prodrugs, carboxylic acid isosteres, N-oxides and S-oxides. Further, in the context of the present invention, reference to the compounds of Formula (I) includes reference to the compounds presented herein in one or more embodiments either as such or represented by one or more structural formula.

The term “optionally substituted” means “substituted or unsubstituted,” and therefore, the generic structural formulae described herein encompasses compounds containing the specified optional substituent as well as compounds that do not contain the optional substituent.

The term “NAFLD” means a chronic liver disease occurring in the absence of alcohol consumption (e.g., <20 gr/day), affecting 2.8-24% of the general population all over the world. It is increasingly diagnosed worldwide and considered to be the commonest liver disorder in Western countries, affecting approximately 15-30% of the general population, and its prevalence increases steadily to 70-90% in people with obesity or type 2 diabetes. It is also currently the most common cause of abnormal liver function and is recognized as a major cause of cryptogenic cirrhosis of liver. The common occurrence of NAFLD in the Asia-Pacific region is partially attributable to the increasing prevalence of the major risk factors for NAFLD like the metabolic syndrome and its individual components. Estimates of its current prevalence range from 5% to 30%, depending on the population studied in that region.

The term “NASH” means a chronic liver disease with inflammation caused by fat accumulation in the absence of alcohol consumption (<20 gr/day). Pathological features of NASH include simple hepatic steatosis and, more characteristically, liver cell damage and accompanying inflammation and/or fibrosis. Steatosis may result from increased fat supply such as high-fat diet from gut to liver, increased influx of free fatty acids from the non-esterified pool, decreased fat export in the form of very low-density lipoprotein-triglyceride, decreased free fatty β-oxidation, and increased de novo lipogenesis (DNL) (Clin Nutr. Vol 33, 2014 pp: 186-190; Gastroenterology. Vol 146, 2014 pp: 726-735). A simple steatosis in 20-30% of NAFLD patients transforms to NASH. Thus, pathogenesis of the NASH is characterized by steatosis, liver inflammation, hepatocellular ballooning, and progressive liver fibrosis. NASH may ultimately lead to cirrhosis and the end-stage liver disease like hepatocellular carcinoma.

### DESCRIPTIVE ASPECTS AND EMBODIMENTS

In one aspect, the compound of General Formula (I) is a compound of General Formula (Ia),

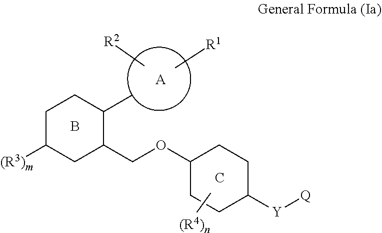

wherein:

Ring A is saturated or unsaturated 4- to 6-membered carbocycle; or 5- to 6-membered heteroaryl; or saturated or partly saturated or unsaturated 5- to 10-membered heterocyclic ring which contains 1, 2, 3 or 4 heteroatoms independently selected from N, O and S;

Ring B is phenyl; or 6-membered heteroaryl which contains 1, 2 or 3 N atoms;

Ring C is phenyl; or 6-membered heteroaryl which contains 1, 2 or 3 N atoms;

Y is —(CR¹⁴R¹⁵)_{g}—; Q is —CO₂M, —CONH₂, —CONH[(C₁-C₆)alkyl], —CON[(C₁-C₆)alkyl]₂ or —CONHSO₂(C₁-C₆)alkyl; M is hydrogen, deuterium or (C₁-C₆)alkyl;

R¹ is

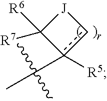

wherein

[symbol] is point of attachment;

J is —CH₂—, —CHF—, —CF₂—, —CH[(C₁-C₆)alkyl], —C[(C₁-C₆)alkyl]₂, —O—, —NR^{a}— or —S—;

“ [symbol] ” represents an optional bond;

R^{a} is hydrogen, (C₁-C₆)alkyl and halo(C₁-C₆)alkyl;

R² is selected from the group consisting of hydrogen, halogen, (C₁-C₆)alkyl, halo(C₁-C₆)alkyl, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl-, heterocyclyl-(C₁-C₆)alkyl-, (C₁-C₆)alkyl-heterocyclyl, heteroaryl-(C₁-C₆)alkyl-, (C₁-C₆)alkyl-heteroaryl-, cyano, —C(O)NR¹⁰R¹¹, —C(S)NR¹⁰R¹¹, —S(O)ₜR¹² and —C(O)R¹³; or

R¹ and R² are combined together with one or two atoms of Ring A to form:

i) a 3- to 8-membered partly saturated or saturated carbocycle; or

ii) a 4- to 8-membered saturated heterocycle which contains 1, 2 or 3 heteroatoms independently selected from the group consisting of N, O and S;

wherein the said carbocycle or the heterocycle can be unsubstituted or substituted with one or more groups independently selected from the group consisting of (C₁-C₆)alkenyl, (C₁-C₆)alkyl, halo(C₁-C₆)alkyl, (C₁-C₆)alkoxy, hydroxy, halogen, cyano, oxo, (C₃-C₁₀)cycloalkyl, (C₆-C₁₀)aryl, heteroaryl and heterocyclyl;

R³, at each occurrence, is independently selected from the group consisting of hydrogen, halogen, (C₁-C₆)alkyl, halo(C₁-C₆)alkyl, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³;

R⁴, at each occurrence, is independently selected from the group consisting of hydrogen, (C₁-C₆)alkyl, halo(C₁-C₆)alkyl, halogen, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —S(O)ₜR¹² and —C(O)R¹³;

R⁵, at each occurrence, is independently selected from the group consisting of hydrogen, (C₁-C₆)alkyl, hydroxy, cyano, —COR¹⁰, —NR¹⁰R¹¹, —CONR¹⁰R¹¹, (C₁-C₆)alkylamino, di(C₁-C₆)alkylamino, (C₁-C₆)alkoxy, halo(C₁-C₆)alkyl, —S(O)ₜR¹² and —C(O)R¹³;

R⁶ and R⁷ are independently selected from the group consisting of hydrogen, (C₁-C₆)alkyl, halo(C₁-C₆)alkyl and halogen;

R¹⁰ is hydrogen, hydroxy, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkylene-, (C₁-C₆)alkyl-(C₆-C₁₀)arylene-, heterocyclyl-(C₁-C₆)alkyl, (C₁-C₆)alkyl-heterocyclyl, heteroaryl-(C₁-C₆)alkyl or (C₁-C₆)alkyl-heteroaryl;

R¹¹ is hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl or —S(O)ₜR¹²;

or

R¹⁰ and R¹¹ are combined together to form 3- to 8-membered saturated or unsaturated ring which contains 1, 2 or 3 heteroatoms independently selected from N, O and S;

R¹² and R¹³ are independently selected from the group consisting of hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl and (C₁-C₆)alkyl-heteroaryl;

R¹⁴ and R¹⁵ are independently selected from hydrogen, (C₁-C₆)alkyl, halo(C₁-C₆)alkyl and halogen; or

R¹⁴ and R¹⁵ are combined together to form a 3- to 5-membered saturated or unsaturated ring which optionally contains 1 or 2 heteroatoms independently selected from N, O and S;
g is 2, 3, 4, 5 or 6;
m is 0, 1 or 2;
n is 0, 1 or 2;
r is 0, 1, 2, 3 or 4;
t is 0, 1 or 2;

wherein

(C₁-C₆)alkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, (C₆-C₁₀)aryloxy, heterocyclyl, heteroaryl, amino, cyano, nitro, —NH(C₁-C₆)alkyl, —N[(C₁-C₆)alkyl]₂, —C(O)(C₁-C₆)alkyl, —C(O)O(C₁-C₆)alkyl, —C(O)NH₂, —C(O)NH(C₁-C₆)alkyl, —C(O)N[(C₁-C₆)alkyl]₂ and —C(O)NHSO₂(C₁-C₆)alkyl;

(C₃-C₁₀)cycloalkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, amino, cyano and nitro;

carbocycle is unsubstituted or substituted with one or more groups independently selected from the group consisting of (C₁-C₆)alkyl, halo(C₁-C₆)alkyl, hydroxy, halogen, (C₁-C₆)alkoxy, halo(C₁-C₆)alkoxy, (C₆-C₁₀)aryl, (C₃-C₁₀)cycloalkyl, heteroaryl, heterocyclyl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;

(C₆-C₁₀)aryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;

heterocyclyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)NR¹⁰R¹¹ and —S(O)ₜR¹²; wherein R¹⁰, R¹¹, R¹² and t are as defined above;

heteroaryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, heterocyclyl, heteroaryl, amino, cyano, nitro, —C(O)NR¹⁰R¹¹ or —S(O)ₜR¹²; wherein R¹⁰, R¹¹, R¹² and t are as defined above;

or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof.

In one aspect, the compound of General Formula (I) is a compound of General Formula (Ib),

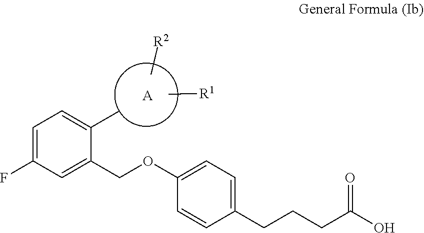

wherein the variables are defined as herein, or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof.

In some embodiments, Ring A is saturated or unsaturated 4- to 6-membered carbocycle. In some embodiments, Ring A is 5- to 6-membered heteroaryl. In some embodiments, Ring A is saturated or partly saturated or unsaturated 5- to 10-membered heterocyclic ring which contains 1, 2, 3 or 4 heteroatoms independently selected from N, O and S.

In some embodiments, Ring A is thiazole, thiophene, thiadiazole, pyridine or pyrimidine. In some embodiments, Ring A is substituted thiazole, substituted thiophene, substituted thiadiazole, substituted pyridine or substituted pyrimidine.

In some embodiments, one of R¹ and R² is hydrogen. In some embodiments, R¹ is cyclopropyl optionally substituted with 1-2 substituents selected from halo, such as fluoro, C₁-C₆ alkyl, haloC₁-C₆ alkyl, cyano. In some embodiments, R¹ is cyclopropyl or cyclobutyl. In some embodiments, R¹ is substituted cyclopropyl.

In some embodiments, R¹ and R² are combined together with one or two atoms of Ring A to form a 4- to 8-membered partly saturated or saturated carbocycle. In some embodiments, the 4- to 8-membered partly saturated or saturated carbocycle is unsubstituted. In some embodiments, the 4- to 8-membered partly saturated or saturated carbocycle is substituted.

In some embodiments, R¹ and R² are combined together with one or two atoms of Ring A to form a 4- to 8-membered saturated heterocycle which contains 1, 2 or 3 heteroatoms independently selected from the group consisting of N, O and S. In some embodiments, the 4- to 8-membered saturated heterocycle is unsubstituted. In some embodiments, the 4- to 8-membered saturated heterocycle is substituted.

A list of non limiting representative compounds according to General Formula (I) utilized herein are disclosed below (the number associated with each refers to the compound's serial number or SI. No.):
1. 4-(4-((2-(5-(1-Cyanocyclopropyl)thiophen-2-yl)-S-fluorobenzyl)oxy)phenyl)butanoic acid;
2. 4-(4-((4-Fluoro-4′-(1-methylcyclopropyl)-[1,1′-biphenyl]-2-yl)methoxy)phenyl)butanoic acid;
3. 4-(4-((4′-(1-Cyanocyclopropyl)-4-fluoro-[1,1′-biphenyl]-2-yl)methoxy)phenyl)butanoic acid;
4. 4-(4-((4′-Cyclopropyl-4-fluoro-[1,1′-biphenyl]-2-yl)methoxy)phenyl)butanoic acid;
5. 4-(4-((2-(2,3-Dihydro-1H-inden-S-yl)-S-fluorobenzyl)oxy)phenyl)butanoic acid;
6. 4-(4-((5-Fluoro-2-(5,6,7,8-tetrahydronaphthalen-2-yl)benzyl)oxy)phenyl)butanoic acid;
7. 4-(4-((2-(Bicyclo[4.2.0]octa-1(6),2,4-trien-3-yl)-S-fluorobenzyl)oxy)phenyl)butanoic acid;
8. 4-(4-((2-(5-Cyclopropylthiophen-2-yl)-S-fluorobenzyl)oxy)phenyl)butanoic acid;
9. 4-(4-((2-(2,3-Dihydrobenzofuran-S-yl)-S-fluorobenzyl)oxy)phenyl)butanoic acid;
10. 4-(4-((5-Fluoro-2-(4,5,6,7-tetrahydrobenzo[b]thiophen-2-yl)benzyl)oxy)phenyl) butanoic acid;
11. 4-(4-((2-(6,7-Dihydro-5H-cyclopenta[b]pyridin-3-yl)-S-fluorobenzyl)oxy)phenyl) butanoic acid;
12. 4-(4-((2-(Bicyclo[4.2.0]octa-1(6),2,4-trien-3-yl)benzyl)oxy)phenyl)butanoic acid;
13. 4-(4-((2-(5-Cyclobutylthiophen-2-yl)-S-fluorobenzyl)oxy)phenyl)butanoic acid;
14. 4-(4-((4′-Cyclopropyl-4-fluoro-3′-methyl-[1,1′-biphenyl]-2-yl)methoxy) phenyl)butanoic acid;
15. 4-(4-((2-(6-Cyclopropylpyridin-3-yl)-S-fluorobenzyl)oxy)phenyl)butanoic acid;
16. 4-(4-((2-(2-Cyclopropylpyrimidin-S-yl)-S-fluorobenzyl)oxy)phenyl)butanoic acid;
17. 4-(4-((2-(2,3-Dihydro-1H-inden-S-yl)benzyl)oxy)phenyl)butanoic acid;
18. 4-(4-((2-(7,8-Dihydronaphthalen-2-yl)-S-fluorobenzyl)oxy)phenyl)butanoic acid;
19. 4-(4-((5-Fluoro-2-(5-methylene-5,6,7,8-tetrahydronaphthalen-2-yl)benzyl)oxy) phenyl) butanoic acid;
20. 4-(4-((5-Fluoro-2-(5-(1-methylcyclopropyl)thiophen-2-yl)benzyl)oxy)phenyl) butanoic acid;
21. 4-(4-((5-Fluoro-2-(5,6,7,8-tetrahydro-4H-cyclohepta[d]thiazol-2-yl)benzyl)oxy) phenyl) butanoic acid;
22. 4-(4-((4′-(2,2-Difluorocyclopropyl)-4-fluoro-[1,1′-biphenyl]-2-yl)methoxy)phenyl) butanoic acid;
23. 4-(4-((2-(5-Cyclopropyl-1,3,4-thiadiazol-2-yl)-S-fluorobenzyl)oxy)phenyl)butanoic acid;
24. 4-(4-((2-(5-Cyclopropylthiazol-2-yl)-S-fluorobenzyl)oxy)phenyl)butanoic acid;
25. 4-(4-((5-Fluoro-2-(4,5,6,7-tetrahydrobenzo[d]thiazol-2-yl)benzyl)oxy)phenyl) butanoic acid;
26. 4-(4-((2-(5,6-Dihydro-4H-cyclopenta[d]thiazol-2-yl)-S-fluorobenzyl)oxy)phenyl) butanoic acid;

The compounds utilized herein can be prepared by art known methods, and modifications thereof, such as those disclosed in US 2016/0347768 corresponding to PCT/IB2015/051232.

It is preferable to formulate the effective compounds in a composition comprising one or more pharmaceutically acceptable carriers or vehicles to administer on human or test animals. The acceptable carriers or vehicles for administering through different routes are well-known in the art. Such routes may be oral, parenteral, transocular, intranasal, transdermal, transmucosal, by inhalation spray, vaginally, rectally, or by intracranial injection.

Pharmaceutically acceptable carriers include any and all clinically useful solvents, dispersion media, coatings, antibacterial and antifungal agents, isotonic and absorption delaying agents and the like. Pharmaceutical carriers also include pharmaceutically acceptable salts, pharmaceutically acceptable esters of the compounds. The compounds may form solvates with water or common organic solvents.

### EXAMPLES

Some representative compounds (referred to as test compounds) of General Formula (I) as provided above, were tested for demonstrating their activity using the assays and the methods described below:

### Example 1. In Vitro Testing

TNFα and MCP-1

Tumor Necrosis Factor-α (TNF-α) is an inflammatory mediator secreted by several inflammatory cell types, including monocyte/macrophages, neutrophils, and T-cells, but also by many other tissues, such as the endothelium, adipose tissue, or neuronal tissue. In the liver, TNF-α is secreted directly by hepatocytes and Kupffer cells or indirectly by abdominal fat. Several studies have shown that TNF-α is a key factor in the development of NAFLD and NASH in both humans and animals.

Monocyte chemotactic protein-1 (MCP-1) is a major chemokine responsible for the recruitment of leukocytes into the liver during hepatic inflammation through the activation of CCR2 receptor displayed on inflammatory cells. MCP-1 levels appear to be increased in NASH patients and in diet-induced NASH models.

The inhibition of TNF-α in targeted myeloid cells prevents the infiltration of monocytes by blocking the production of MCP-1, lessening the progression of NASH.

Assay for TNFα and MCP-1 Release in RAW 264.7 Cells:

Cells were seeded at a density of 50000 cells/well/100 μl in a 96 well plate and left overnight in the CO₂ incubator under normal cell culture conditions for adherence. Next day, old media was removed and fresh media added per well with compounds in duplicate for 1 hour with 0.1% DMSO. Cells were stimulated with LPS (Lipopolysaccharides) at 10 ng/ml and incubated at cell culture conditions for 2.5 hours. Post incubation, the supernatant was collected in fresh 96 well plate and stored at −80 degree for TNF-α, and MCP-1 estimation.

The dose-response curve was analyzed using Sigma Plot/Graph Pad. The IC₅₀ (concentration of the test compounds where 50% of compounds' maximal activity is observed) values were calculated from the dose-response curve.

The following compounds were selected for the above assays.
SI.2: 4-(4-((4-Fluoro-4′-(1-methylcyclopropyl)-[1,1′-biphenyl]-2-yl)methoxy) phenyl) butanoic acid;

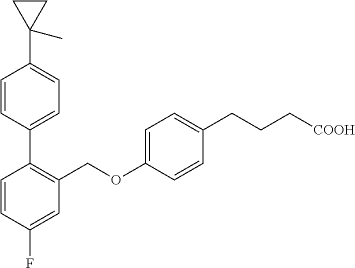 [SMILES: CC1(c2ccc(-c3ccc(F)cc3COc3ccc(CCCC(=O)O)cc3)cc2)CC1]

SI.8: 4-(4-((2-(5-Cyclopropylthiophen-2-yl)-S-fluorobenzyl)oxy) phenyl) butanoic acid-metformin salt

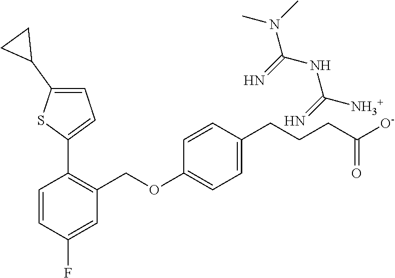

SI.20: 4-(4-((5-Fluoro-2-(5-(1-methylcyclopropyl)thiophen-2-yl)benzyl)oxy) phenyl) butanoic acid (metformin salt);

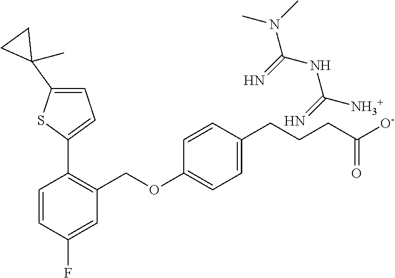

SI.24: 4-(4-((2-(5-Cyclopropylthiazol-2-yl)-S-fluorobenzyl) oxy) phenyl) butanoic acid;

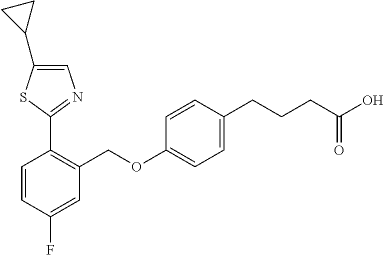 [SMILES: O=C(O)CCCc1ccc(OCc2cc(F)ccc2-c2ncc(C3CC3)s2)cc1]

SI.25: 4-(4-((5-Fluoro-2-(4,5,6,7-tetrahydrobenzo[d]thiazol-2-yl)benzyl) oxy) phenyl) butanoic acid

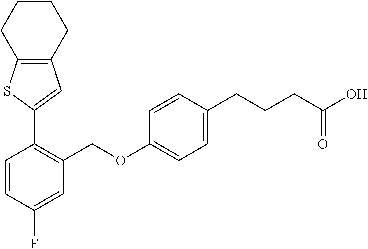 [SMILES: O=C(O)CCCc1ccc(OCc2cc(F)ccc2-c2cc3c(s2)CCCC3)cc1]

The results of in vitro TNFα and MCP-1 assay are provided in the table below.

| | | TNF-alpha | MCP-1 |
|---|---|---|---|
| S. | | IC50 | IC50 |
| No | Compound Name | (nM) | (nM) |
| 2 | 4-(4-((4-Fluoro-4′-(1-methylcyclopropyl)-[1,1′- | 5650 | 290 |
| | biphenyl]-2-yl)methoxy)phenyl)butanoic acid; | | |
| 8 | 4-(4-((2-(5-Cyclopropylthiophen-2-yl)-5-fluoro- | >10000 | 2040 |
| | benzyl)oxy)phenyl)butanoic acid-metformin | | |
| | salt | | |
| 20 | 4-(4-((5-Fluoro-2-(5-(1-methylcyclopropyl) | 660 | 1080 |
| | thiophen-2-yl)benzyl)oxy)phenyl) butanoic | | |
| | acid-metformin salt; | | |
| 24 | 4-(4-((2-(5-Cyclopropylthiazol-2-yl)-5- | 630 | 230 |
| | fluorobenzyl)oxy)phenyl)butanoic acid; | | |
| 25 | 4-(4-((5-Fluoro-2-(4,5,6,7-tetra- | >10000 | >10000 |
| | hydrobenzo[d]thiazol-2- | | |
| | yl)benzyl)oxy)phenyl) butanoic acid; | | |

### Example 2. In Vivo Testing

The model of diet induced obesity in mice by chronic administration of high fat diet (HFD, 60% kcal for periods extending more than 4-6 months) results in the development of obesity, insulin resistance, inflammation and fibrosis. This model mimics certain basic features of NASH found in clinical setting in a milder form. Clinically, development of steatosis is followed by progression to NASH in up to one-third of patients with NAFLD. NASH is diagnosed when hepatocellular steatosis occurs with concurrent necroinflammatory reactions of the liver and hepatocellular ballooning with or without fibrosis and/or cirrhosis. Lobular inflammation (usually in acinar zone 3) and portal inflammation are both present in NASH. Lobular inflammation is followed by infiltration of affected areas by innate immune cells. Portal inflammation is common and usually mild. Other histological lesions present in NASH include hepatocellular ballooning, fibrosis, apoptotic bodies, sinusoidal collagen formation, Mallory-Denk bodies (MDBs), megamitochondria, glycogenated nuclei, and iron deposition.

In accordance with the present invention, animals fed 60% HFD for a period of more than 6 months were treated with compounds—
(i) SI.8: 4-(4-((2-(5-Cyclopropylthiophen-2-yl)-S-fluorobenzyl)oxy) phenyl) butanoic acid-metformin salt, and
(ii) SI. 25:4-(4-((5-Fluoro-2-(4,5,6,7-tetrahydrobenzo[d]thiazol-2-yl) benzyl) oxy)phenyl) butanoic acid,

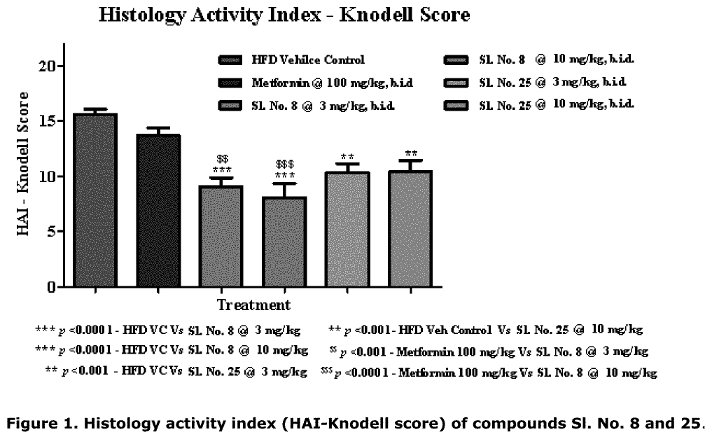

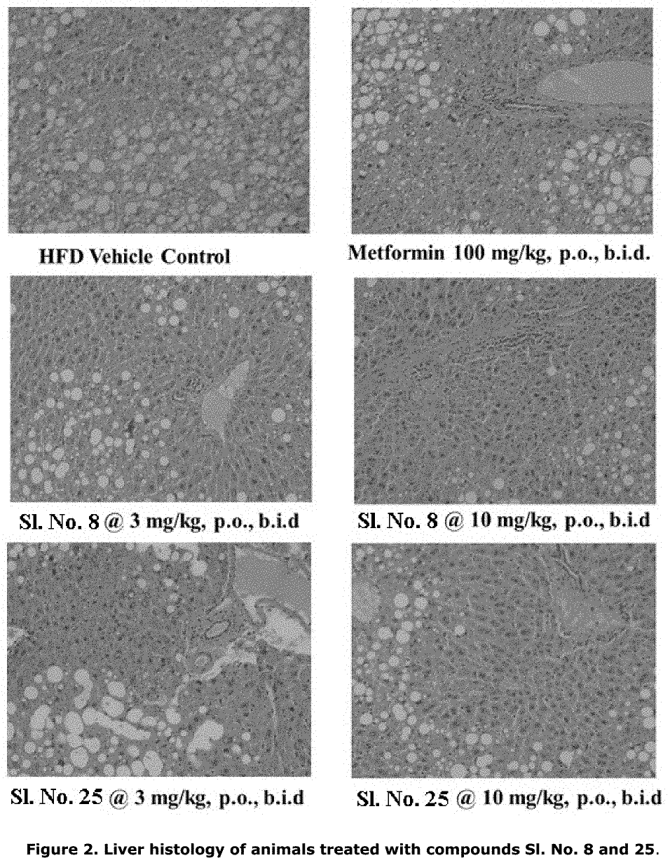

for about 8 weeks and histology of liver samples was performed to demonstrate the efficacy of the compounds. Histologic features of NASH include macro-steatosis, ballooning, and lobular inflammation. Knodell histological activity index (HAI-Knodell) was estimated to evaluate the effect of compounds on NASH. There was substantial improvement of HAI (FIG. 1). Compound treatment resulted in reduction in ballooning and microvascular necrosis, resulting in the regeneration of hepatocytes (FIG. 2, H&E staining, 20×). Recovery of hepatocytes towards normal morphology following compound treatment demonstrated the positive effect of the compounds in the treatment of NASH and NASH like symptoms, as provided herein.

The present invention is described with the help of some embodiments and non limiting examples and various modifications are possible without departing from the scope of the disclosure and the claims.

1. A method of treating a subject suffering from non-alcoholic steatohepatitis (NASH), non-alcoholic fatty liver disease (NAFLD), steatosis, lobular inflammation, or liver fibrosis, or slowing the progression of NAFLD to NASH in a subject in need thereof, or reducing liver inflammation in a subject suffering from NASH, comprising administering to said subject a therapeutically effective amount of at least one of the compounds of General Formula (I) or a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere thereof; 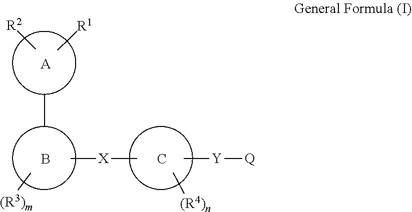
wherein:
Ring A is a saturated or unsaturated 4- to 10-membered carbocycle; a 5- to 10-membered heteroaryl; or a saturated or partly saturated or unsaturated 5- to 10-membered heterocycle;
wherein said heteroaryl or heterocycle contain 1, 2, 3 or 4 heteroatoms independently selected from N, O and S;
Ring B and Ring C are independently selected from the group consisting of (C₆-C₁₀)aryl and 6- to 10-membered heteroaryl which contains 1, 2 or 3 heteroatoms independently selected from the group consisting of N, O and S;
X is —(CR⁸R⁹)ₚ—O—, —(CR⁸R⁹)ₚ—S—, —(CR⁸R⁹)ₚ—N(R¹⁰)—, —O—(CR⁸R⁹)ₚ—, —S—(CR⁸R⁹)ₚ— or —N(R¹⁰)—(CR⁸R⁹)ₚ;
Y is —(CR¹⁴R¹⁵)_{g}—;
Q is —CO₂M, —CONH₂, —CONH[(C₁-C₆)alkyl], —CON[(C₁-C₆)alkyl]₂ or —CONHSO₂(C₁-C₆)alkyl;
M is hydrogen, deuterium or (C₁-C₆)alkyl;
R¹ is 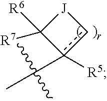
wherein [symbol]
is point of attachment to Ring A;
J is —CH₂—, —CHF—, —CF₂—, —CH[(C₁-C₆)alkyl], —C[(C₁-C₆)alkyl]₂, —O—, —NR^{a}— or —S—;
“ [symbol] ” represents an optional bond;
R^{a} is hydrogen, (C₁-C₆)alkyl or halo(C₁-C₆)alkyl;
R² is selected from the group consisting of hydrogen, halogen, (C₁-C₆)alkyl, halo(C₁-C₆)alkyl, (C₁-C₆)alkoxy, hydroxy, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl-, (C₆-C₁₀)aryloxy, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³; or
R¹ and R² are combined together with one or two atoms of Ring A to form:
i) a 3- to 8-membered partly unsaturated or saturated carbocycle; or
ii) a 4- to 8-membered saturated heterocycle which contains 1, 2 or 3 heteroatoms independently selected from the group consisting of N, O and S;
wherein the said carbocycle or heterocycle can be unsubstituted or substituted with the one or more groups independently selected from the group consisting of (C₁-C₆)alkenyl, (C₁-C₆)alkyl, halo(C₁-C₆)alkyl, (C₁-C₆)alkoxy, hydroxy, halogen, cyano, oxo, (C₃-C₁₀)cycloalkyl, (C₆-C₁₀)aryl, heteroaryl and heterocyclyl;
R³ at each occurrence, is independently selected from the group consisting of hydrogen, halogen, (C₁-C₆)alkyl, halo(C₁-C₆)alkyl, (C₁-C₆)alkoxy, hydroxy, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, (C₆-C₁₀)aryloxy, heterocyclyl-(C₁-C₆)alkyl, (C₁-C₆)alkyl-heterocyclyl-, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³;
R⁴ at each occurrence, is independently selected from the group consisting of hydrogen, (C₁-C₆)alkyl, halo(C₁-C₆)alkyl, (C₁-C₆)alkoxy, hydroxy, halogen, cyano, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, (C₆-C₁₀)aryloxy, heterocyclyl-(C₁-C₆)alkyl, (C₁-C₆)alkyl-heterocyclyl, heteroaryl-(C₁-C₆)alkyl, (C₁-C₆)alkyl-heteroaryl, —NR¹⁰R¹¹, —S(O)ₜR¹² and —C(O)R¹³;
R⁵ is selected from the group consisting of hydrogen, (C₁-C₆)alkyl, hydroxy, cyano, —COR¹⁰, —NR¹⁰R¹¹, —CONR¹⁰R¹¹, (C₁-C₆)alkylamino, di(C₁-C₆)alkylamino, (C₁-C₆)alkoxy, halo(C₁-C₆)alkyl, —S(O)ₜR¹² and —C(O)R¹³;
R⁶ and R⁷ are independently selected from the group consisting of hydrogen, (C₁-C₆)alkyl, halo(C₁-C₆)alkyl and halogen;
R⁸ and R⁹ are independently selected from the group consisting of hydrogen, deuterium, (C₁-C₆)alkyl, halo(C₁-C₆)alkyl and halogen; or
R⁸ and R⁹ can combine together to form:
i) a 3- to 5-membered saturated carbocycle selected from the group consisting of cyclopropane, cyclobutane, cyclopentane and cyclohexane; or
ii) a 4- to 6-membered saturated heterocycle selected from the group consisting of oxetane, thietane, azetidine, tetrahydrofuran, tetrahydrothiophene, pyrolidine and piperidine;
R¹⁰ is hydrogen, hydroxy, (C₁-C₆)alkyl, halo(C₁-C₆)alkyl, (C₁-C₆)alkoxy, (C₃-C₁₀)cycloalkyl, (C₆-C₁₀)aryl, heterocyclyl, heteroaryl, (C₆-C₁₀)aryloxy, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl or (C₁-C₆)alkyl-heteroaryl or —S(O)ₜR¹²;
R¹¹ is hydrogen, hydroxy, (C₁-C₆)alkyl, halo(C₁-C₆)alkyl, (C₃-C₁₀)cycloalkyl, (C₁-C₆)alkoxy, (C₃-C₁₀)cycloalkyl, (C₆-C₁₀)aryl, heterocyclyl, heteroaryl, (C₆-C₁₀)aryloxy, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl or —S(O)ₜR¹²; or
R¹⁰ and R¹¹ are combined together to form 3- to 8-membered saturated or unsaturated ring which contains 1, 2 or 3 heteroatoms independently selected from N, O and S;
R¹² and R¹³ are independently selected from the group consisting of hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl and (C₁-C₆)alkyl-heteroaryl;
R¹⁴ and R¹⁵ are independently selected from the group consisting of hydrogen, (C₁-C₆)alkyl, halo(C₁-C₆)alkyl and halogen; or
R¹⁴ and R¹⁵ are combined together to form a 3- to 5-membered saturated carbocycle or 4- to 6-membered saturated heterocycle which optionally contains 1 or 2 heteroatoms independently selected from the group consisting of N, O and S; wherein the said carbocycle or heterocycle can be unsubstituted or substituted;
g is 2, 3, 4, 5 or 6;
m is 0, 1 or 2;
n is 0, 1 or 2;
p is 1, 2 or 3;
r is 0, 1, 2, 3 or 4;
t is 0, 1 or 2;
wherein
(C₁-C₆)alkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, (C₆-C₁₀)aryloxy, heterocyclyl, heteroaryl, amino, cyano, nitro, —NH(C₁-C₆)alkyl, —N[(C₁-C₆)alkyl]₂, —C(O)(C₁-C₆)alkyl, —C(O)O(C₁-C₆)alkyl, —C(O)NH₂, —C(O)NH(C₁-C₆)alkyl, —C(O)N[(C₁-C₆)alkyl]₂ and —C(O)NHSO₂(C₁-C₆)alkyl;
(C₃-C₁₀)cycloalkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, amino, cyano and nitro;
carbocycle is unsubstituted or substituted with one or more groups independently selected from the group consisting of (C₁-C₆)alkyl, halo(C₁-C₆)alkyl, hydroxy, halogen, (C₁-C₆)alkoxy, halo(C₁-C₆)alkoxy, (C₆-C₁₀)aryl, (C₃-C₁₀)cycloalkyl, heteroaryl, heterocyclyl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;
(C₆-C₁₀)aryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;
heterocyclyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)NR¹⁰R¹¹ and —S(O)ₜR¹²; wherein R¹⁰, R¹¹, R¹² and t are as defined above;
heteroaryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, heterocyclyl, heteroaryl, amino, cyano, nitro, —C(O)NR¹⁰R¹¹ and —S(O)ₜR¹²;
wherein R¹⁰, R¹¹, R¹² and t are as defined above.

2. The method as claimed in claim 1, wherein the compound of General Formula (I) is a compound of General Formula (Ia): 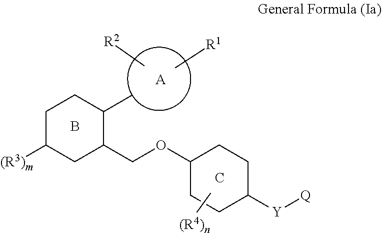
wherein:
Ring A is saturated or unsaturated 4- to 6-membered carbocycle; or 5- to 6-membered heteroaryl; or saturated or partly saturated or unsaturated 5- to 10-membered heterocyclic ring which contains 1, 2, 3 or 4 heteroatoms independently selected from N, O and S;
Ring B is phenyl; or 6-membered heteroaryl which contains 1, 2 or 3 N atoms;
Ring C is phenyl; or 6-membered heteroaryl which contains 1, 2 or 3 N atoms;
Y is —(CR¹⁴R¹⁵)_{g}—; Q is —CO₂M, —CONH₂, —CONH[(C₁-C₆)alkyl], —CON[(C₁-C₆)alkyl]₂ or —CONHSO₂(C₁-C₆)alkyl; M is hydrogen, deuterium or (C₁-C₆)alkyl;
R¹ is 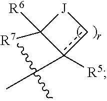
wherein [symbol]
is point of attachment;
J is —CH₂—, —CHF—, —CF₂—, —CH[(C₁-C₆)alkyl], —C[(C₁-C₆)alkyl]₂, —O—, —NR^{a}— or —S—;
“ [symbol] ” represents an optional bond;
R^{a} is hydrogen, (C₁-C₆)alkyl and halo(C₁-C₆)alkyl;
R² is selected from the group consisting of hydrogen, halogen, (C₁-C₆)alkyl, halo(C₁-C₆)alkyl, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl-, heterocyclyl-(C₁-C₆)alkyl-, (C₁-C₆)alkyl-heterocyclyl, heteroaryl-(C₁-C₆)alkyl-, (C₁-C₆)alkyl-heteroaryl-, cyano, —C(O)NR¹⁰R¹¹, —C(S)NR¹⁰R¹¹, —S(O)ₜR¹² and —C(O)R¹³; or
R¹ and R² are combined together with one or two atoms of Ring A to form:
iii) a 3- to 8-membered partly saturated or saturated carbocycle; or
iv) a 4- to 8-membered saturated heterocycle which contains 1, 2 or 3 heteroatoms independently selected from the group consisting of N, O and S;
wherein the said carbocycle or the heterocycle can be unsubstituted or substituted with one or more groups independently selected from the group consisting of (C₁-C₆)alkenyl, (C₁-C₆)alkyl, halo(C₁-C₆)alkyl, (C₁-C₆)alkoxy, hydroxy, halogen, cyano, oxo, (C₃-C₁₀)cycloalkyl, (C₆-C₁₀)aryl, heteroaryl and heterocyclyl;
R³, at each occurrence, is independently selected from the group consisting of hydrogen, halogen, (C₁-C₆)alkyl, halo(C₁-C₆)alkyl, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —C(O)NR¹⁰R¹¹, —C(S)NR¹⁰R¹¹, —S(O)ₜR¹² and —C(O)R¹³;
R⁴, at each occurrence, is independently selected from the group consisting of hydrogen, (C₁-C₆)alkyl, halo(C₁-C₆)alkyl, halogen, (C₃-C₁₀)cycloalkyl, (C₆-C₁₀)aryl, heteroaryl, heterocyclyl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl, cyano, —NR¹⁰R¹¹, —S(O)ₜR¹² and —C(O)R¹³;
R⁵, at each occurrence, is independently selected from the group consisting of hydrogen, (C₁-C₆)alkyl, hydroxy, cyano, —COR¹⁰, —NR¹⁰R¹¹, —CONR¹⁰R¹¹, (C₁-C₆)alkylamino, di(C₁-C₆)alkylamino, (C₁-C₆)alkoxy, halo(C₁-C₆)alkyl, —S(O)ₜR¹² and —C(O)R¹³;
R⁶ and R⁷ are independently selected from the group consisting of hydrogen, (C₁-C₆)alkyl, halo(C₁-C₆)alkyl and halogen;
R¹⁰ is hydrogen, hydroxy, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkylene-, (C₁-C₆)alkyl-(C₆-C₁₀)arylene-, heterocyclyl-(C₁-C₆)alkyl, (C₁-C₆)alkyl-heterocyclyl, heteroaryl-(C₁-C₆)alkyl or (C₁-C₆)alkyl-heteroaryl;
R¹¹ is hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl, (C₁-C₆)alkyl-heteroaryl or —S(O)ₜR¹²;
or
R¹⁰ and R¹¹ are combined together to form 3- to 8-membered saturated or unsaturated ring which contains 1, 2 or 3 heteroatoms independently selected from N, O and S;
R¹² and R¹³ are independently selected from the group consisting of hydrogen, (C₁-C₆)alkyl, halo(C₁-C₆)alkyl, (C₃-C₁₀)cycloalkyl, (C₆-C₁₀)aryl, heterocyclyl, heteroaryl, (C₆-C₁₀)aryl-(C₁-C₆)alkyl, (C₁-C₆)alkyl-(C₆-C₁₀)aryl, heterocyclyl-(C₁-C₆)alkyl, (C₁-C₆)alkyl-heterocyclyl, heteroaryl-(C₁-C₆)alkyl and (C₁-C₆)alkyl-heteroaryl;
R¹⁴ and R¹⁵ are independently selected from hydrogen, (C₁-C₆)alkyl, halo(C₁-C₆)alkyl and halogen; or
R¹⁴ and R¹⁵ are combined together to form a 3- to 5-membered saturated or unsaturated ring which optionally contains 1 or 2 heteroatoms independently selected from N, O and S;
g is 2, 3, 4, 5 or 6;
m is 0, 1 or 2;
n is 0, 1 or 2;
r is 0, 1, 2, 3 or 4;
t is 0, 1 or 2;
wherein
(C₁-C₆)alkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, (C₆-C₁₀)aryloxy, heterocyclyl, heteroaryl, amino, cyano, nitro, —NH(C₁-C₆)alkyl, —N[(C₁-C₆)alkyl]₂, —C(O)(C₁-C₆)alkyl, —C(O)O(C₁-C₆)alkyl, —C(O)NH₂, —C(O)NH(C₁-C₆)alkyl, —C(O)N[(C₁-C₆)alkyl]₂ and —C(O)NHSO₂(C₁-C₆)alkyl;
(C₃-C₁₀)cycloalkyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, amino, cyano and nitro;
carbocycle is unsubstituted or substituted with one or more groups independently selected from the group consisting of (C₁-C₆)alkyl, halo(C₁-C₆)alkyl, hydroxy, halogen, (C₁-C₆)alkoxy, halo(C₁-C₆)alkoxy, (C₆-C₁₀)aryl, (C₃-C₁₀)cycloalkyl, heteroaryl, heterocyclyl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;
(C₆-C₁₀)aryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)O(C₁-C₆)alkyl, —C(O)NR¹⁰R¹¹ and —S(O)ₜR¹²; wherein R¹⁰, R¹¹, R¹² and t are as defined above;
heterocyclyl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, (C₃-C₁₀)cycloalkyl, (C₆-C₁₀)aryl, heterocyclyl, heteroaryl, amino, cyano, nitro, —C(O)NR¹⁰R¹¹ and —S(O)ₜR¹²; wherein R¹⁰, R¹¹, R¹² and t are as defined above;
heteroaryl is unsubstituted or substituted with one or more groups independently selected from the group consisting of (C₁-C₆)alkyl, halogen, halo(C₁-C₆)alkyl, hydroxy, (C₁-C₆)alkoxy, halo(C₁-C₆)alkoxy, heterocyclyl, heteroaryl, amino, cyano, nitro, —C(O)NR¹⁰R¹¹ or —S(O)ₜR¹²;
wherein R¹⁰, R¹¹, R¹² and t are as defined above;
or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof.

3. The method as claimed in claim 1, wherein the compound of General Formula (I) is a compound of General Formula (Ib), 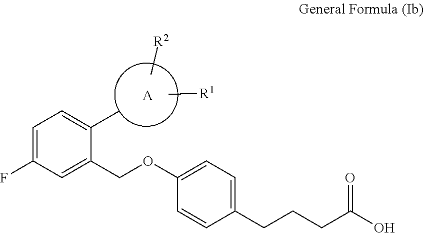
wherein the variables are defined as in claim 1.

4. The method as claimed in claim 1, wherein the compound is:
4-(4-((2-(5-(1-Cyanocyclopropyl)thiophen-2-yl)-S-fluorobenzyl)oxy)phenyl)butanoic acid;
4-(4-((4-Fluoro-4′-(1-methylcyclopropyl)-[1,1′-biphenyl]-2-yl)methoxy)phenyl)butanoic acid;
4-(4-((4′-(1-Cyanocyclopropyl)-4-fluoro-[1,1′-biphenyl]-2-yl)methoxy)phenyl)butanoic acid;
4-(4-((4′-Cyclopropyl-4-fluoro-[1,1′-biphenyl]-2-yl)methoxy)phenyl)butanoic acid;
4-(4-((2-(2,3-Dihydro-1H-inden-S-yl)-S-fluorobenzyl)oxy)phenyl)butanoic acid;
4-(4-((5-Fluoro-2-(5,6,7,8-tetrahydronaphthalen-2-yl)benzyl)oxy)phenyl)butanoic acid;
4-(4-((2-(Bicyclo[4.2.0]octa-1(6),2,4-trien-3-yl)-S-fluorobenzyl)oxy)phenyl)butanoic acid;
4-(4-((2-(5-Cyclopropylthiophen-2-yl)-S-fluorobenzyl)oxy)phenyl)butanoic acid;
4-(4-((2-(2,3-Dihydrobenzofuran-S-yl)-S-fluorobenzyl)oxy)phenyl)butanoic acid;
4-(4-((5-Fluoro-2-(4,5,6,7-tetrahydrobenzo[b]thiophen-2-yl)benzyl)oxy) phenyl)butanoic acid;
4-(4-((2-(6,7-Dihydro-5H-cyclopenta[b]pyridin-3-yl)-S-fluorobenzyl)oxy)phenyl) butanoic acid;
4-(4-((2-(Bicyclo[4.2.0]octa-1(6),2,4-trien-3-yl)benzyl)oxy)phenyl)butanoic acid;
4-(4-((2-(5-Cyclobutylthiophen-2-yl)-S-fluorobenzyl)oxy)phenyl)butanoic acid;
4-(4-((4′-Cyclopropyl-4-fluoro-3′-methyl-[1,1′-biphenyl]-2-yl)methoxy) phenyl)butanoic acid;
4-(4-((2-(6-Cyclopropylpyridin-3-yl)-S-fluorobenzyl)oxy)phenyl)butanoic acid;
4-(4-((2-(2-Cyclopropylpyrimidin-S-yl)-S-fluorobenzyl)oxy)phenyl)butanoic acid;
4-(4-((2-(2,3-Dihydro-1H-inden-S-yl)benzyl)oxy)phenyl)butanoic acid;
4-(4-((2-(7,8-Dihydronaphthalen-2-yl)-S-fluorobenzyl)oxy)phenyl)butanoic acid;
4-(4-((5-Fluoro-2-(5-methylene-5,6,7,8-tetrahydronaphthalen-2-yl)benzyl)oxy) phenyl) butanoic acid;
4-(4-((5-Fluoro-2-(5-(1-methylcyclopropyl)thiophen-2-yl)benzyl)oxy)phenyl) butanoic acid;
4-(4-((5-Fluoro-2-(5,6,7,8-tetrahydro-4H-cyclohepta[d]thiazol-2-yl)benzyl)oxy) phenyl) butanoic acid;
4-(4-((4′-(2,2-Difluorocyclopropyl)-4-fluoro-[1,1′-biphenyl]-2-yl)methoxy)phenyl) butanoic acid;
4-(4-((2-(5-Cyclopropyl-1,3,4-thiadiazol-2-yl)-S-fluorobenzyl)oxy)phenyl)butanoic acid;
4-(4-((2-(5-Cyclopropylthiazol-2-yl)-S-fluorobenzyl)oxy)phenyl)butanoic acid;
(4-((5-Fluoro-2-(4,5,6,7-tetrahydrobenzo[d]thiazol-2-yl)benzyl)oxy)phenyl) butanoic acid;
4-(4-((2-(5,6-Dihydro-4H-cyclopenta[d]thiazol-2-yl)-S-fluorobenzyl)oxy)phenyl) butanoic acid;
or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof.

5. The method as claimed in claim 1, wherein the compound of General Formula (I) is administered as a pharmaceutical composition.

6. The method as claimed in claim 3, wherein the composition further comprises at least one known compound with complimentary mechanism such as metformin, thiazolidinedione, PPAR alpha/delta agonists, FXR agonists PPAR alpha/gamma agonists.

7. The method as claimed in claim 3, wherein the composition is administered orally.

8. The method as claimed in claim 3, wherein the composition is administered parenterally.

9. A method of diagnosing and treating non-alcoholic steatohepatitis (NASH) in a patient, said method comprising:
a. obtaining a lever tissue sample from a human patient;
b. detecting concurrent necroinflammatory reactions of the liver and hepatocellular ballooning with or without fibrosis and/or cirrhosis by way of biopsy;
c. diagnosing the patient suffering from NASH when concurrent necroinflammatory reactions of the liver and hepatocellular ballooning with or without fibrosis and/or cirrhosis are detected; and
d. administering an effective amount of compound of General Formula (I) or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof to the diagnosed patient.

10. Compounds of General Formula (I) or a stereoisomer, a tautomer, a geometrical isomer, a pharmaceutically acceptable salt, a pharmaceutically acceptable solvate, a prodrug, a polymorph, an N-oxide, a S-oxide or a carboxylic acid isostere thereof for use in the treatment of a patient suffering from non-alcoholic steatohepatitis (NASH), or another disorder disclosed herein.

11. Use of a compound of General Formula (I) or a stereoisomer, a tautomer or a geometrical isomer thereof or a pharmaceutically acceptable salt, a pharmaceutically acceptable solvate, a prodrug, a polymorph, an N-oxide, a S-oxide or a carboxylic acid isostere thereof for preparation of a medicament for treatment of non-alcoholic steatohepatitis (NASH), or another disorder disclosed herein.
